(19) United States
(12) Patent Application Publication
(10) Pub. No.: US 2005/0089546 A1
(43) Pub. Date: Apr. 28, 2005

(54) Compositions and methods comprising complex carbohydrate and alpha-lactalbumin

(76) Inventors: Dick Wurtman, Boston, MA (US); Judy Wurtman, Boston, MA (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/939,967

(22) Filed: Jan. 3, 2005

Related U.S. Application Data
(60) Provisional application No. 60/502,968, filed on Sep. 16, 2003.

(51) Int. Cl. A61K 38/38; A61K 31/715

(52) U.S. Cl. 424/439; 514/12; 514/54

(57) ABSTRACT

The present invention provides compositions and formulations comprising alpha-lactalbumin and a carbohydrate of high glycemic index. This invention also provides methods of treating pain, pre-menstrual syndrome, and symptoms associated with the pre-menstrual period and menopause.

### CROSS-REFERENCE TO RELATED APPLICATION

This application claims priority from U.S. Provisional Patent Application 60/502,968, filed Sep. 16, 2003, which is hereby incorporated by reference.

### FIELD OF THE INVENTION

The present invention provides nutritional compositions and formulations for consumption by humans or animals. This invention also provides methods for treating or ameliorating pre-menstrual syndrome, menopause, or associated symptoms thereof, and for treating or ameliorating pain.

### BACKGROUND OF THE INVENTION

The premenstrual phase of the menstrual cycle may produce a variety of mood, appetite and pain symptoms that usually disappear with the onset of menses. Some symptoms may appear mid-cycle around the time of ovulation but typically are most prominent a few days before the onset of menses. These symptom complaints can be clustered into categories: dysphoric mood, increased appetite, pain and discomfort, sleep disturbances, difficulty with work and social functioning.

The neurotransmitter serotonin (5-hydroxytryptamine or 5-HT) has been shown to mediate the mood and appetite symptoms of PMS and treatment with carbohydrate rich dietary interventions that increase brain serotonin has been shown to improve these symptoms. Recently, specific serotonin reuptake inhibitors, like fluoxetine, have been shown to be effective in improving the more severe mood disturbances (4-6) and even the physical symptoms of PMS, essentially confirming that serotonin is the likely mechanism of action for the effects found with the carbohydrates.

It has been noted, however, that physical complaints and mood disturbances are also common during much of the luteal phase, or second half, of the menstrual cycle. These symptoms include breast tenderness, bloating, headache and joint or muscle pain, especially in the lower back. As Steiner et al demonstrated, interventions altering serotonin may also diminish pain and physical discomfort.

The neurotransmitter serotonin (5-hydroxytryptamine or 5-HT) is 3-(beta-aminoethyl)-5-hydroxyindole. Serotonin can be formed in the body by hydroxylation and decarboxylation of the essential amino acid L-tryptophan. In the biosynthesis of serotonin from L-tryptophan, L-tryptophan is hydroxylated in the presence of the enzyme tryptophan hydroxylase to form the intermediate product L-5-hydroxytryptophan (L-5-HTP). This intermediate product is decarboxylated in the presence of the enzyme 5-hydroxytryptophan decarboxylase to form serotonin.

Serotonin is present in highest concentration in blood platelets and in the gastrointestinal tract, where it is found in the enterochromaffin cells and the myenteric plexis. Lesser amounts are found in the brain, particularly in the hypothalamus. Serotonin is also found in relatively high concentrations in the lateral gray horns of the spinal cord and in a number of areas in the brain. It can be shown that there is a system of serotonin-containing neurons that have their cell bodies in the raphe nuclei of the brain stem and project to portions of the hypothalamus, the limibic system, the neocortex, and the spinal cord.

Serotonin stimulates or inhibits a variety of smooth muscles and nerves and, among others, has effects on secretion by both exocrine and endocrine glands and on functioning of the respiratory, cardiovascular and central nervous systems. Within the central nervous system (CNS), serotonin serves as a neurotransmitter in the brain and spinal cord, where it is a chemical transmitter of neurons referred to as tryptaminergic or serotonergic neurons. These neurons are involved in control of sleep, appetite, nutrient selection, blood pressure, mood, endocrine secretion, aggressivity and numerous other sensitivities to external stimuli.

### SUMMARY OF THE INVENTION

The invention provides a composition comprising an amount of a carbohydrate, which has a high glycemic index, and an amount of alpha-lactalbumin protein. In another embodiment, the invention provides a composition comprising about up to 50 grams of a carbohydrate, which has a high glycemic index, and about 1-20 grams of alpha-lactalbumin protein. In another embodiment, the invention provides a formulation comprising an amount of a carbohydrate, which has a high glycemic index, and an amount of alpha-lactalbumin protein. In another embodiment, the invention provides a formulation comprising about up to 50 grams of a carbohydrate which has a high glycemic index, and about 1-20 grams of alpha-lactalbumin. In another embodiment, the invention provides a method of treating pain comprising the steps of administering a composition comprising an amount of a carbohydrate, which has a high glycemic index, and an amount of alpha-lactalbumin protein, thereby treating said pain. In another embodiment, the invention provides a method of ameliorating pain comprising the steps of administering a composition comprising an amount of a carbohydrate, which has a high glycemic index, and an amount of alpha-lactalbumin protein, thereby treating said pain. In another embodiment, the invention provides a method of treating pain associated with pre-menstrual syndrome, comprising the steps of administering a composition comprising an amount of a carbohydrate which has a high glycemic index, and an amount of alpha-lactalbumin protein, thereby treating said pain. In another embodiment, the invention provides a method of ameliorating pain associated with pre-menstrual syndrome, comprising the steps of administering a composition comprising an amount of a carbohydrate, which has a high glycemic index, and an amount of alpha-lactalbumin protein, thereby treating said pain. In another embodiment, the invention provides a method of treating pain associated with menopause, comprising the steps of administrating an amount of a carbohydrate, which has a high glycemic index, and an amount of alpha-lactalbumin protein, thereby treating said treating pain associated with menopause. In another embodiment, the invention provides a method of ameliorating pain associated with menopause, comprising the steps of administrating an amount of a carbohydrate, which has a high glycemic index, and an amount of alpha-lactalbumin protein, thereby treating said treating pain associated with menopause. In another embodiment, the invention provides a method of treating increased appetite associated with pre-menstrual syndrome, comprising the steps of administrating an amount of a carbohydrate, which has a high glycemic index, and an amount of alpha-lactalbumin protein, thereby treating increased appetite associated with pre-menstrual syndrome. In another embodiment, the invention provides a method of ameliorating increased appetite associated with pre-menstrual syndrome, comprising the steps of administrating an amount of a carbohydrate, which has a high glycemic index, and an amount of alpha-lactalbumin protein, thereby treating increased appetite associated with pre-menstrual syndrome. In another embodiment, the invention provides a method of treating mood symptoms associated with pre-menstrual syndrome comprising the steps of administrating an amount of a carbohydrate which has a high glycemic index, and an amount of alpha-lactalbumin protein, thereby treating mood symptoms associated with pre-menstrual syndrome. In another embodiment, the invention provides a method of ameliorating mood symptoms associated with pre-menstrual syndrome comprising the steps of administrating an amount of a carbohydrate which has a high glycemic index, and an amount of alpha-lactalbumin protein, thereby treating mood symptoms associated with pre-menstrual syndrome. In another embodiment, the invention provides a method of ameliorating pre-menstrual syndrome, comprising the steps of administrating an amount of a carbohydrate, which has a high glycemic index, and an amount of alpha-lactalbumin protein, thereby ameliorating pre-menstrual syndrome. In another embodiment, the invention provides a method of treating insomnia associated with pre-menstrual syndrome, comprising the steps of administrating an amount of a carbohydrate which has a high glycemic index, and an amount of alpha-lactalbumin protein, thereby treating insomnia associated with pre-menstrual syndrome.

### BRIEF DESCRIPTION OF THE DRAWINGS

FIG. 1 depicts the ethnic/minority composition of the sample from which the subjects for the amino acid level study (A) and the PMS study (B) were drawn.

FIG. 2 depicts the number of observations per subject for each intervention combination.

FIG. 3 presents an analysis showing the rise in tryptophan induced by alpha-lac as compared to C+C at different time points following intervention. (A) treatment/baseline ratios of plasma tryptophan level between the alpha-lac and C+C groups; (B) Mean differences, standard errors, and p values of the ratios in (A). (C) Differences between the “high” and “low” alpha-lac groups.

FIG. 4 presents an analysis showing the rise in tryptophan induced by alpha-lac as compared to carbohydrates alone at different time points following intervention.

FIG. 5 presents an analysis showing the treated/baseline ratio of tryptophan/LNAA ratio induced by casein+carbohydrate, carbohydrate alone, and low and high alpha-lac+carbohydrate (A). Alpha-lac is compared individually to both C+C at different time points following intervention (B). Low and High alpha-lac are compared to one another (C). Alpha-lac is compared to and carbohydrates alone (D).

### DETAILED DESCRIPTION OF THE INVENTION

The present invention provides, in one embodiment, compositions or formulations comprising alpha-lactalbumin protein (alpha-lac) and a carbohydrate that has a high glycemic index (high GI carbohydrate). In another embodiment, the compositions or formulations comprise up to about 50 grams of said high GI carbohydrate. In another embodiment, the compositions or formulations comprise up to about 45 grams of said high GI carbohydrate. In another embodiment, the compositions or formulations comprise up to about 40 grams of said high GI carbohydrate. In another embodiment, the compositions or formulations comprise up to about 35 grams of said high GI carbohydrate. In another embodiment, the compositions or formulations comprise up to about 32.5 grams of said high GI carbohydrate.

In another embodiment, the compositions or formulations comprise between about 5 and 50 grams of said high GI carbohydrate. In another embodiment, the compositions or formulations comprise between about 10 and 50 grams of said high GI carbohydrate. In another embodiment, the compositions or formulations comprise between about 15 and 50 grams of said high GI carbohydrate. In another embodiment, the compositions or formulations comprise between about 20 and 50 grams of said high GI carbohydrate. In another embodiment, the compositions or formulations comprise between about 25 and 50 grams of said high GI carbohydrate. In another embodiment, the compositions or formulations comprise between about 30 and 50 grams of said high GI carbohydrate. In another embodiment, the compositions or formulations comprise between about 32.5 and 50 grams of said high GI carbohydrate. In another embodiment, the compositions or formulations comprise between about 35 and 50 grams of said high GI carbohydrate. In another embodiment, the compositions or formulations comprise about 32.5 grams of said high GI carbohydrate. In another embodiment, the compositions or formulations comprise about 35 grams of said high GI carbohydrate.

In another embodiment, the compositions or formulations comprise between about 1 and 20 grams of said alpha-lac. In another embodiment, the compositions or formulations comprise between about 2 and 20 grams of said alpha-lac. In another embodiment, the compositions or formulations comprise between about 4 and 20 grams of said alpha-lac. In another embodiment, the compositions or formulations comprise between about 6 and 20 grams of said alpha-lac. In another embodiment, the compositions or formulations comprise between about 8 and 20 grams of said alpha-lac. In another embodiment, the compositions or formulations comprise between about 1 and 16 grains of said alpha-lac. In another embodiment, the compositions or formulations comprise between about 1 and 12 grams of said alpha-lac. In another embodiment, the compositions or formulations comprise between about 1 and 8 grams of said alpha-lac. In another embodiment, the compositions or formulations comprise between about 1 and 6 grams of said alpha-lac. In another embodiment, the compositions or formulations comprise between about 1 and 4 grams of said alpha-lac. In another embodiment, the compositions or formulations comprise about 4 grams of said alpha-lac. In another embodiment, the compositions or formulations comprise about 8 grams of said alpha-lac.

In one embodiment, the compositions or formulations are in the form of an extruded bar. In one embodiment, the compositions or formulations are in the form of a powder. In one embodiment, the compositions or formulations are in the form of a food or beverage. In one embodiment, the compositions or formulations are in the form of a pharmaceutical composition.

In one embodiment, the alpha-lac and high GI carbohydrate are mixed, blended, or combined together. In another embodiment, they are administered together without prior mixing. In another embodiment, they are administered separately.

If the composition or formulation is suitable for oral administration, said composition or formulation may contain, in addition to the active ingredient, additives such as: starch e.g. potato, maize or wheat starch or cellulose or starch derivatives such as microcrystalline cellulose; silica; various sugars such as lactose; magnesium carbonate and/or calcium phosphate. It is desirable that, if the oral formulation is for administration it will be well tolerated by the patient's digestive system. To this end, it may be desirable to include in the formulation mucus formers and resins. It may also be desirable to improve tolerance by formulating the composition or formulations in a capsule which is insoluble in the gastric juices. It may also be preferable to include the composition or formulation in a controlled release formulation.

If the composition or formulation is suitable for rectal administration the formulation may contain a binding and/or lubricating agent; for example polymeric glycols, gelatins, cocoa butter or other vegetable waxes or fats.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Alternatively, the composition or formulation may be in dry form, for reconstitution before use with an appropriate sterile liquid.

In addition to the active ingredients, these pharmaceutical compositions may contain suitable pharmaceutically acceptable carriers comprising excipients and auxiliaries, which facilitate processing of the active compounds into preparations, which can be used pharmaceutically. Further details on techniques for formulation and administration may be found in the latest edition of Remington's Pharmaceutical Sciences (Maack Publishing, Easton Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combining active compounds with solid excipients and processing the resultant mixture of granules (optionally, after grinding) to obtain tablets or dragee cores. Suitable auxiliaries can be added, if desired. Suitable excipients include carbohydrate or protein fillers, such as sugars, including lactose, sucrose, mannitol, and sorbitol; starch from corn, wheat, rice, potato, or other plants; cellulose, such as methyl cellulose, hydroxypropylmethyl-cellulose, or sodium carboxymethylcellulose; gums, including arabic and tragacanth; proteins, such as gelatin and collagen, flavoring agents, coloring agents and coating materials (e.g., wax or a plasticizer). A composition to be administered orally, but in liquid form, can include, optionally, an emulsifying agent, a flavoring agent and/or a coloring agent. If desired, disintegrating or solubilizing agents may be added, such as the cross-linked polyvinyl pyrrolidone, agar, and alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations, which can be used orally, include push-fit capsules made of gelatin, as well as soft, sealed capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms.

After pharmaceutical compositions have been prepared, they can be placed in an appropriate container and labeled for treatment of an indicated condition.

The compounds of this invention include pharmaceutically acceptable derivatives of the alpha-lac and high-GI carbohydrates and, as applicable, pharmaceutically acceptable ammonium salts thereof. A “pharmaceutically acceptable derivative” means any pharmaceutically acceptable salt, ester, or salt of such ester, of a compound of this invention or any other compound which, upon administration to a recipient, is capable of providing (directly or indirectly) a compound of this invention or an active metabolite or residue thereof.

Salts derived from appropriate bases include alkali metal (e.g., sodium), alkaline earth metal (e.g., magnesium), ammonium and N—(C.sub. 1-4 alkyl).sub.4.sup.+salts.

The compounds of this invention contain one or more asymmetric carbon atoms and thus may occur as racemates and racemic mixtures, single enantiomer, diastereomeric mixtures and individual diastereoisomers. All such isomeric forms of these compounds are expressly included in the present invention. Each stereogenic carbon may be of the R or S configuration.

Pharmaceutically acceptable salts of the compounds of this invention include those derived from pharmaceutically acceptable inorganic and organic acids and bases. Examples of such acid salts include: acetate, adipate, alginate, aspartate, benzoate, benzenesulfonate, bisulfate, butyrate, citrate, camphorate, camphorsulfonate, cyclopentanepropionate, digluconate, dodecylhydrogensulfate, dodecylsulfate, ethanesulfonate, formate, fumarate, glucoheptanoate, glycerophosphate, glycollate, hemisulfate, heptanoate, hexanoate, hydrochloride, hydrobromide, hydroiodide, 2-hydroxyethanesulfonate, lactate, maleate, malonate, methanesulfonate, 2-naphthylsulfonate, nicotinate, nitrate, oxalate, pamoate, pectinate, perchlorate, persulfate, 3-phenylpropionate, phosphate, picrate, pivalate, propionate, salicylate, succinate, sulfate, tartrate, thiocyanate, tosylate, and undecanoate.

This invention also provides, in one embodiment, the quaternization of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chlorides, bromides and iodides; dialkyl sulfates including dimethyl, diethyl, dibutyl and diamyl sulfates; long chain halides such as decyl, lauryl, myristyl and stearyl cldorides, bromides and iodides, and aralkyl halides including benzyl and phenethyl bromides. Water or oil-soluble or dispersible products may be obtained by such quaternization.

Other materials, which may optionally be included in the nutritional supplement of the present invention include B-complex vitamins. Also, ingredients such as sweeteners, flavorants, coloring agents, dyes, preservatives, emulsifying agents, suspending agents, melting agents, excipients, and solvents or diluents such as water, ethanol, propylene glycol, glycerin and various combinations thereof, may be included in the nutritional supplement of the present invention.

In one embodiment, this invention provides sweeteners which may be used in the nutritional supplement of the present invention include, but are not limited to, saccharin, aspartame, cyclamates, acesulfame K, neohesperidin dihydrochalcone, other super sweeteners, and mixtures thereof, which may be added to the carrier in amounts sufficiently low so as not to chemically interact with the main ingredients of the nutritional supplement.

The flavorants which may be used in the nutritional supplement of the present invention-include, but are not limited to, peppermint, peppermint-menthol, eucalyptol wintergreen, licorice, clove, cinnamon, spearmint, cherry, lemon, orange lime, menthol and various combinations thereof.

The compositions of this invention can also be administered through sachets to which the subject adds water, or as a food based preparation, functional food, dietary supplement or nutraceutical. In one embodiment, “functional food” is a food engineered or supplemented to give improved nutritional value. In one embodiment, “dietary supplement” is a substance produced by isolation, or microbial culture purification that gives health benefits. In one embodiment, “nutraceutical” is a food, or parts of a food, that provide medical or health benefits, including prevention and treatment of clinical conditions and/or symptoms related thereto. The compositions of this invention can also be isolated from varying plants or components thereof including but not limited to root, tuber, rind/peel, bark, seed, fruit, bulb, flower, rhizome, leaf, stem, oil, shell, capsule, twig, resin, extract, and bean. In addition, the aforementioned components can be consumed by the subject, thereby providing the subject with the active ingredient(s) of the invention disclosed herein.

It is to be understood that those skilled in the art of pharmaceutical formulation will be able to make a variety of formulations that would be within the scope of this disclosure and the appended claims, without departing from the spirit and teachings of the invention. It is intended that all such formulations be included in this invention.

In one embodiment, the term “alpha-lac” refers to all naturally occurring or synthetic derivatives or isoforms of alpha lac and proteins that are homologous to bovine alpha-lac from any species, including cows, provided that said proteins have a tryptophan content of greater than about 2% tryptophan Such proteins may include, for example, macadamia nut protein and pichia pastoris yeast protein (U.S. Pat. No. 4,709,449). Said alpha-lac may be isolated or purified from any biological source or may be produced by recombinant methods. Said alpha-lac may be in the form of whey protein, whey protein concentrate, whey powder, or alpha-lac-enriched whey protein. Methods for producing alpha-lac-enriched whey protein are known in the art (see U.S. Pat. No. 6,312,755).

In one embodiment, the term “carbohydrate” or “carbohydrates” refers to any carbohydrates that are customary in the preparation of foods, such as ingestible monossaccharidic or dissaccharidic materials, their hydrolysis products, and mixtures thereof, for example, dextrose (glucose), sucrose, fructose, lactose, maltose, galactose, sugar alcohols, such as sorbitol, mannitol and xylitol, invert sugar syrups, brown sugar, corn syrup, corn syrup solids, honey, molasses, maple syrup, and the like, which are commercially available from sources known by those of skill in the art, and mixtures thereof. In one embodiment, a single carbohydrate is used. In another embodiment, a mixture of more than one carbohydrate is used.

In one embodiment, the compositions or formulations are utilized in ameliorating or preventing symptoms that occur prior to onset of menstruation (pre-menstrual period). In another embodiment, said symptoms may occur in the luteal phase of the menstrual cycle. In another embodiment, the present invention provides a method of treating or ameliorating said symptoms. Said symptoms may be pain, discomfort, unease, increased appetite, excessive appetite, craving for carbohydrates, mood changes, mood disruptions, mood fluctuations, dysphoria, insomnia, sleep disturbances, exhaustion, muscle aches and cramps, or any other symptoms or disturbances that may accompany the pre-menstrual period or the luteal phase of the menstrual cycle. In one embodiment, said symptoms comprise those listed in the Diagnostic and Statistical Manual of Mental Disorders, Fourth Edition, (DSM IV) published by the American Psychiatric Association.

In one embodiment, the pre-menstrual period is the period of time beginning 3-7 days prior to the onset of menses. In another embodiment, the pre-menstrual period begins approximately 4 days prior to the onset of menses. In another embodiment, the pre-menstrual period is the period of time beginning approximately 3 days prior to the onset of menses. In another embodiment, the pre-menstrual period is the period of time beginning approximately 2 days prior to the onset of menses. In another embodiment, the pre-menstrual period is the period of time beginning approximately 1 day prior to the onset of menses. In one embodiment, the pre-menstrual period extends until the onset of menstruation. In another embodiment, the pre-menstrual period extends until the end of menstruation. In another embodiment, the pre-menstrual period extends until 7 days following the onset of menstruation. In another embodiment, the pre-menstrual period encompasses the second half of the menstrual cycle.

In another embodiment, said compositions or formulations are utilized to ameliorate pre-menstrual syndrome (PMS). In one embodiment, PMS refers to discomfort, pain, or unease, physical or emotional, associated with the pre-menstrual period, in any of its manifestations, as described hereinabove. In another embodiment, PMS is described in the Diagnostic and Statistical Manual of Mental Disorders, Fourth Edition, (DSM IV). In another embodiment, the present invention provides methods of ameliorating PMS or said manifestations.

In general, the composition of the present invention is administered to an individual prior to the expected onset of her menstrual period. The length of time during which the composition is administered varies on an individual basis, but in general will be from 1 to 14 days prior to onset of menstruation and might continue (e.g., 3 days) after its onset. The amount of the composition administered daily will also vary on an individual basis and to some extent will be determined by the type and severity of symptoms to be treated.

The compositions and formulations of the present invention act to increase plasma levels of tryptophan, plasma ratio of tryptophan to large neutral amino acids (LNAA), or both (Examples 2-4). Without limiting this invention to a particular mechanism, increases in plasma levels of tryptophan or plasma tryptophan/LNAA ratios are believed to increase serotonin levels in the brain. Since serotonin is believed to be involved in the symptoms accompanying pre-menstrual syndrome (PMS), increasing brain serotonin levels is a method of treating these symptoms.

In another embodiment, said compositions or formulations are utilized to ameliorate pain or discomfort resulting from or accompanying menopause.

In another embodiment, said compositions or formulations are utilized to ameliorate pain. In another embodiment, the current invention provides a method for treatment or amelioration of pain. In one embodiment, the pain is chronic pain. In another embodiment, the pain is chronic pain.

Pain is a complex subjective phenomenon comprised of a sensation indicating real or potential tissue damage and the affective response this generates. Pain can be classified as either acute or chronic pain. Acute pain is an essential biologic signal of the potential for or the extent of injury. It is usually short-lived and is associated with hyperactivity of the sympathetic nervous system; eg, tachycardia, increased respiratory rate and blood pressure, diaphoresis, and pupillary dilation. The concurrent affect is anxiety. Treatment involves removal of the underlying etiology if possible and the use of analgesic drugs.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells or in animal models such as mice, rats, rabbits, dogs, or pigs. An animal model may also be used to determine the appropriate concentration range and route of administration. Such information can then be used to determine useful doses and routes for administration in humans. Effective dose is determined also in clinical trials in humans.

The effective amount of the nutritional supplement will vary depending on such factors as the patient being treated, the particular mode of administration, the activity of the particular active ingredients employed, the age, bodyweight, general health, sex and diet of the patient, time of administration, rate of excretion, the particular combination of ingredients employed, the total content of the main ingredient of the nutritional supplement, the severity of the illness or symptom, and the result sought. It is within the skill of the person of ordinary skill in the art to account for these factors. In one embodiment, the effective amount of the composition or formulation will be determined based on the factors mentioned hereinabove. In another embodiment, the quantities of alpha-lac and high GI carbohydrate will each be determined separately on an individual basis.

In one embodiment, said alpha-lac and high-GI carbohydrate can be administered in a single dose. In another embodiment, they can be administered in a number of smaller doses over a period of time. In another embodiment, one or more components can be administered in a single dose, while the other component(s) can be administered in smaller doses.

The invention will now be described through illustrative examples. The examples are not intended to limit the scope of the invention, which is limited only by the appended claims.

### EXAMPLES

### Materials and Methods

### Plasma Amino Acid Level Study (Examples 1-4)

Experimental Design and Methods:

This study investigated whether taking 4 or 8 g. of purified alpha-lactalbumin, with 35 g. of a dextrose/dextrin/maltodextrin mixture that enhances insulin secretion, increases the plasma tryptophan ratio. The study was carried out using a 4-period, 4 treatment crossover design; carbohydrates (48.5 g.) were tested alone, the carbohydrates (32.5 g.) plus 16 g. of tryptophan-poor protein (i.e. casein) and the carbohydrates (35 g) with 4 or 8 g. of purified alpha-lactalbumin. The carbohydrate was in the form of a raspberry powdered drink that is mixed with water; the proteins were added to this drink.

The alpha-lactalbumin was provided by Davisco Foods, a milk processing company. The compound was divided into 4 g. portions, in keeping with CRC standard operating procedures, and 0, 1, or 2 portions was mixed with carbohydrates and water. Casein was similarly divided in 16 g. portions and mixed with the carbohydrates and water. Test meals were weighed and packaged individually by a Research Dietician.

Eight, normal-weight-for-height females between the ages of 21-65 were studied. Potential subjects were screened as follows: Inclusion criteria were females with a BMI between 20-27 and in good health; exclusion criteria included a history of diabetes, a history of milk allergy, late pregnancy, lactating women or the use of hormones or drugs used to treat depression, seizures, or migraines.

Qualified subjects were asked to come to the CRC to read and sign a consent form (discussed with a research staff member) of which they were given a copy. At that time, they were asked to give a medical history and to undergo a physical examination.

The MIT Clinical Research Center outpatient department was the site of the study. If approved, subjects made individual appointments for their test visit. Each subject received each of the four treatments, in randomized order. Test days were separated by at least 2 days after the most recent study day. On the test days, subjects reported to the CRC at 7:15 am after an overnight fast. During the test visits, the subject had vital signs (BP and Pulse) taken and had an angiocath inserted for the first blood drawing. Each of the 4 test mixtures was then administered with water, bloods were again drawn at 1, 2, 3, and 6 hours after its ingestion. Each blood sample was 5 ml., for a total volume of 25 ml. An angiocath with a saline lock was used for the first 4 blood drawings and was then removed. The last blood sample required an additional needle stick. A standardized lunch was provided after the 3 hour blood sample is drawn. The subjects could leave the CRC after lunch with the restriction that they were restricted from eating or drinking anything except for water and could not undertake any heavy exercise during their time away from the CRC prior to coming back for their 6 hour blood sample.

Test meals were weighed and packaged individually by a Research Dietician; the subject consumed the entire contents of the prepared mixture on each test day.

Test Formula

| | Alpha-Lact- | Carbohydrate | Casein |
|---|---|---|---|
| | albumin(gm.) | Mixture (gm.) | (gm.) |
| 1. | — | 48.5 | — |
| 2. | — | 32.5 | 16.0 |
| 3 | 4.0 | 35.0 | — |
| 4 | 8.0 | 35.0 | — |

Blood samples were blinded and batched for analysis. They were spun at 1500×g for 15 minutes at 4 C and the serum was measured for tryptophan and large neutral amino acids by different high performance liquid chromatography (HPLC) methods. Insulin was measured using Diagnostic Products Corp. (DPC) insulin coat-a-count RIA kit.

Primary and Secondary Endpoints and/or Outcomes:

The primary endpoint in this study is the increase (or decrease) in the plasma tryptophan ratio. The secondary endpoints are the increase or decrease in plasma tryptophan levels; plasma tyrosine levels; the plasma tyrosine ratio; and plasma insulin levels.

Biostatistical Analysis:

This study involves two control meals (A: carbohydrates (48.6 g), and B: carbohydrates (32.5 g) and casein (16 g.) and two treatment meals (C: 4 g and D: 8 g alpha-lactalbumin) both given with 35 g. dextrose/dextrin/maltodextrin used as carbohydrates in the control arms.

These four diets were given in a four-period crossover design with 8 subjects. The meals were given in successive periods according to the following 8 sequences: BACD, DCAB, CDAB, DCBA, ABCD, ABDC, CDBA, BADC. Each subject was assigned one of these sequences at random.

Characteristics of the Subject Population:

Eight, normal-weight-for-height females between the ages of 21-65 were studied. Potential subjects were screened as follows: Inclusion criteria were females with a Body Mass Index between 20-27 and in good health; exclusion criteria included a history of diabetes, a history of milk allergy, late pregnancy, lactating women or the use of hormones or drugs used to treat depression seizures, or migraines.

Inclusion of Women and Minorities:

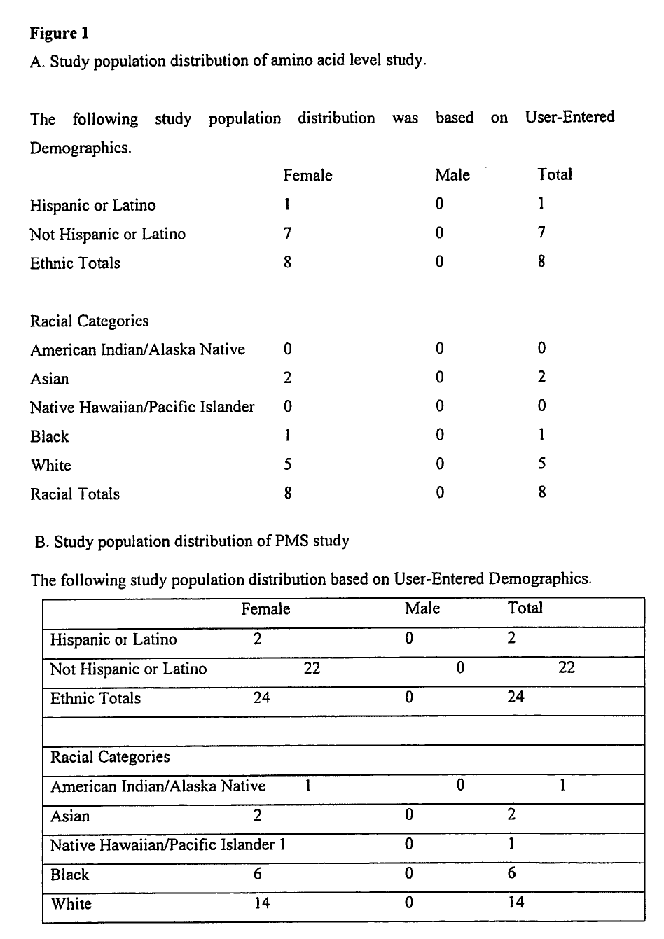

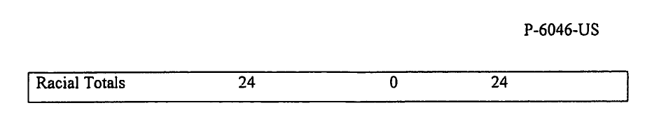

These are depicted in FIG. 1B.

Justification and References: MISER September 2000, 1998 Massachusetts Population estimates, by Age, Gender and Race.

Criteria for Involvement or Exclusion:

The basic physiologic tests performed in this study were not expected, at least initially, to be influenced by ethnicity. Previous studies suggest that the range of responses of female subjects will be similar.

Recruitment:

To recruit subjects representing a diverse population, flyers were placed throughout the University and surrounding areas. In addition, an advertisement was placed in a popular newspaper that reaches the general population in Boston which includes populations at risk such as blacks and Hispanics as will as white subjects.

PMS Study (Examples 5)

Experimental Design and Methods:

This study tested which of the following interventions produces the greatest reduction in the physical complaints associated with PMS in comparison with the placebo. A three menstrual cycle, three treatment double-blind, cross-over study was conducted. The treatment conditions (not in this sequence) were: Carbohydrate Beverage+Casein (C+C)−Carbohydrate Beverage (CHO)−Carbohydrate Beverage+8 gm Alpha-lactalbumin (AP-LB).

Treatment order was balanced for period and carry-over effects across treatments, so subjects were randomized to one of the following 6 treatment orders:
Carbohydrate+Casein, Carbohydrate Drink, Carbohydrate Drink+alpha-lactalbumin
Carbohydrate+Casein, Carbohydrate Drink+alpha-lactalbumin, Carbohydrate Drink
Carbohydrate Drink, Carbohydrate Drink+alpha-lactalbumin, Carbohydrate+Casein
Carbohydrate Drink+alpha-lactalburnin, Carbohydrate Drink, Carbohydrate+Casein
Carbohydrate Drink, Carbohydrate+Casein, Carbohydrate Drink+alpha-lactalbumin
Carbohydrate Drink+alpha-lactalbumin, Carbohydrate+Casein, Carbohydrate Drink

The subjects previously participated in a study surveying the prevalence of premenstrual pain symptoms. Subjects who participated in the PMS symptoms monitoring protocol had kept a daily symptom rating diary (DSR) through two complete menstrual cycles in order to record the frequency and occurrence of mood, appetite and physical symptoms that had been associated with their PMS. They also recorded the onset and duration of their menses. This protocol was approved by COUHES (COUHES No. 2994; MIT CRC No. 522: Survey of Women with Premenstrual Syndrome.

Women whose DSR records indicated mild to severe mood, appetite and/or pain symptoms (for example: cramps, breast tenderness and muscle aches) during the luteal (the last half of the menstrual cycle, ending just before menstruation starts) but not the follicular phase (the first half) of the menstrual cycle were identified and invited to enroll in this beverage study. (Women participating in COUHES No. 2995, CRC No. 522 were told in the consent form that they might be asked to participate in a treatment study of premenstrual symptoms and were asked to indicate their willingness to be solicited for this study. Women who did not consent to be asked to participate in this protocol will not be asked to do so). Women were excluded from the study if they had a history of diabetes or a history of milk allergy. Women will also be excluded if they are night-shift workers. The sample of women from whom the subjects will be drawn, i.e. the women being monitored for the severity and prevalence of premenstrual symptoms represent women who are not pregnant or lactating, using drugs for depression, seizures, or migraines. The subjects for this study were from women who completed two months of DSR records during the monitoring protocol, and had a relatively precise estimate of subjects' anticipated onset of menses.

Qualified subjects came to the MIT CRC to read, sign a consent form (discussed with a research staff member) and were given a copy of the consent. At this visit, the nursing staff took their vital signs (BP and Pulse) and weight in street clothing and the assigned physician or nurse practitioner did a history and physical. Upon approval, a research member set up an appointment for the subject to come in at the beginning of her menstrual cycle. This may be during the last few days of menses, but no later than 7 days post menses. Visits 1-4 were all scheduled during this part of the menstrual cycle. Each visit took approximately 20 minutes with the exception of visit 1, which due to time spent on phone instruction, took approximately 30 minutes.

During the first visit, a research staff member instructed the subject on how to call in to the toll-free automated telephone reporting system and how to respond to the questions. In addition, the subject was instructed on how to mix the test beverage and when to start taking it. The nursing staff took vital signs and the dietary staff supplied one week's (7 packets) worth of test beverage. Extra beverages were supplied in the event that menses came later than 4 days after starting use. Each month for the duration of the study (visits 2-4), the subject came in during her follicular phase for a brief visit in order to return any unused beverages and to pick up a new supply. During these visits, the subject checked in with the nurses to have vital signs taken and to meet with a research staff member to discuss adherence to the testing schedule and record any unforeseen side effects and/or problems.

The subject began the Diary System Rating (DSR-1) on the evening of visit 1 and continued until she had completed 3 full menstrual cycles, possibly four if there was irregularity in one of the cycles. Subjects telephoned the study's toll-free automated phone system each evening and were given a menu-controlled, voice prompts that guided them through an automated data collection system. Subjects were assigned an ID and PIN number to be used when calling in that prompted the caller to press a number key in response to the questions asked. The subject was instructed to call into the telephone system each evening with the exception of when they are taking the test beverage; at this time, additional calls were made before and after taking the intervention. There were 2 different sets of recordings (DSR-1 and SDSR) depending on the subjects' response to the first question, “Are you getting ready to take the test product now?” If the subject keyed in yes, the system queued in the SDSR, otherwise an answer of no directed the subject to the DSR-1.

The telephone prompted the subjects to rate themselves on a variety of mood, pain, appetite and quality of life factors each day during three menstrual cycles (approximately 80-100 days in total). The symptoms are listed in question form and answered by indicating a numerical equivalent for not at all, mild, moderate or severe. The questions characterize the presence and severity of symptoms known to be associated with premenstrual syndrome, such as discomfort and/or pain from breast tenderness, muscular aches, headaches, cramps and bloating. They were also asked to keep track of changes in their mood, appetite and sleep.

The efficacy of the beverages was measured as much as possible without disrupting the subjects' routine and took place in their own, naturalistic environments. Late in the subjects' luteal phase (approximately 4 days prior to menses), subjects were called by a research member the day before the first day of beverage consumption to review the procedures for the test days and to set up an appointment for their next CRC visit. On the assigned test days, the subject ate a normal lunch no later than 12:30 p.m. The test beverage was consumed 2 hours after eating lunch, at approximately 2:30 pm. Prior to consuming the test beverage, the subject called the automated telephone system and gave her study identification number. After indicating by menu selection that she is about to take the test beverage, she was diverted to a series of questions about her pain, mood and appetite (SDRS). At the end of this session, the subject was instructed by the system to consume the test beverage and to call back in one hour, at approximately 3:30 pm. At the time of the second call, the subject was asked the same questions as prior to consumption. This sequence of calling and questioning was repeated again at three hours post-beverage consumption (approximately 5:30 pm). Until data collection is completed after the third call-in, subjects were instructed not to eat and were asked to restrict fluid consumption to non-caffeinated, non-nutritive beverages such as water, soda water or non-caffeinated herbal tea. Because of intra- and inter-subject variability in the timing of menstrual onset, the premenstrual monitoring period was not always exactly 3 days. As such, subjects were asked to continue taking the test beverage and to call in their timed SDSR until the onset of menses. In the event of an early menses and no test days were achieved, the subject continued through one additional menstrual cycle in the hopes of obtaining accurate test data. Any test beverages that were not consumed were returned to the CRC after the end of each month and prior to obtaining the next month's supply of test beverage.

Subjects continued to report their premenstrual symptoms in the evening of each test days as well, using the DSR-1. These data were transferred and secured in the same database and may be used for later comparisons and to correct for any life events that may be unrelated to menstrual symptoms but that may be creating alterations in mood, appetite or physical discomfort.

Daily Symptom Rating Scale (DSR)

There are 3 versions of the DSR:
1. The original version (DSR) that was used in COUHES No. 2994; MIT CRC No. 522.
2. The revised version of the DSR (DSR-1) that was completed every night of the study, including days when taking the intervention;
3. The short version of the DSR-1 (SDSR), which uses just questions 1-18 and was completed three times per day when taking the intervention: once before taking the drink and at two time intervals after taking the drink (1 and 3 hours after consumption).
   Test Beverage:

The test beverage was given in the form of a powder that was mixed with 8 oz. of water. The beverage is raspberry flavored.

The carbohydrate being used is made from potato starch. The alpha-lactalbumin and casein are made from milk products.

Primary and Secondary Endpoints and/or Outcomes:

The primary endpoint in this study is the decrease (or increase) in Pain and Mood Symptoms and analysis includes the pre- and the two post-drink time periods. Food cravings are a secondary end-point and have been analyzed at the pre- and first post-drink time period to decrease confounding by hunger and dinner hour.

Biostatistical Analysis:

Sample Size. The power of the proposed design is based only on an estimated 50% of subjects who were anticipated to complete all three periods of testing. Thus, the study had 12 subjects for any pairwise comparison. As the analysis (described below) includes data from patients who do not complete all three testing periods, it is anticipated that there will be higher power to detect the differences described (or, alternatively, equivalent power to detect smaller differences), especially as have multiple measurements for subjects over time. A very conservative assumption that the test-retest results within a subject would be as variable as the DSR in the total population (Freeman et al, Psych Res 65: 97). Thus, the present study has more power to detect differences than described here. Using a simple paired t-test, 12 subjects provide 80% power to detect a 0.9 within-subject standard deviation (p=0.05, two-sided). Using the population SD of 65.2 previously reported, an 80% power to detect a treatment effect of either the CHO beverage or the CHO beverage+AP LB treatment (compared to each other or CAS) of approximately 59 points (without adjustment for multiple tests) Since the change in the DSR between follicular and luteal phases (the “PMS” effect) is approximately 120 points, then had over 80% power to detect a 50% reduction in symptoms under these very conservative assumptions.

Analysis. A mixed models analysis of variance for the analysis to incorporate repeated measures within a subject was used. Approximately 8-16 measurements during treatment testing for each subject for each month, from four measurements per day for each of the 2-4 days prior to the initiation of menses was used. Of these four measurements, the first measurement (prior to the administration of the test intervention) was entered as a baseline value for the day. An AR (1) correlation structure, with separation was measured in hours. This means that the the measurements within a day could be highly correlated, but that measurements from separate days would effectively be virtually independent, after adjustment for the baseline measurement for the day. Treatment was modeled as two variables (CHO: yes or no; dose of AP-LB: 0 or 8 gms). The effects of the intervention during the menses and carry-over effects were also examined in the analyses models, but these results are considered exploratory.

One complication in the analysis is that on some days women did not have significant symptoms prior to treatment, so that it was not be possible to test whether the treatment has an effect. Therefore, the data was analyzed once with all days included in the analysis, and once limiting the analysis to those days in which a subject has demonstrable PMS symptoms prior to treatment. The results of this second analysis are considered exploratory.

The carbohydrate drink is a 35 g mixture of dextrose, dextrin, maltodextrin and starch. The inactive beverage contains 23 g of the same carbohydrate mixture and 12 g of casein (CAS). The carbohydrates are in the form of a raspberry powdered drink that is mixed with 8 ounces of water. The alpha-lac is also in the form of a powder that is added to the carbohydrate mixture.

The alpha-lac was provided by Davisco Foods, a milk processing company. The test compounds were prepared individually by the BioNutrition Staff, and subjects were instructed on how to mix these compounds with water. Sport water bottles made of plastic were provided to subjects to increase the ease of beverage preparation.

Characteristics of the Subject Population:

Subjects were adult women (age: 21-45) in general good health who have regular menstrual cycles and are not perimenopausal. They were not lactating or pregnant. Subjects were drawn from a pool of subjects monitored throughout two menstrual cycles within the last 3 months.

Ethnic/Minority Composition of the Sample

The sample from which the subjects are drawn for this study are the women who have participated in the monitoring of their premenstrual symptoms. The ethnic/minority composition of this sample is as follow:

| | |
|---|---|
| Caucasian | 57% |
| Black | 29% |
| Hispanic | 6.5% |
| Native American | 1.6% |
| Asian | 5.9% |

Minorities Composition.

Depicted in FIG. 1B.

Justification and References: The sample from which the subjects are drawn for this study are the women who have participated in COUHES protocol 2994; CRC NO. 522. This is the ethnic/minority composition of this sample.

Criteria for Involvement or Exclusion:

Women of all races and ethnicities were invited to participate in COUHES No. 2995, CRC No. 522.

Source of Research Material:

The source of the research material will be obtained through data collected via an automated telephone system. Subjects will be prompted through several questions (addendum 2) and will respond with the press of a numerical key that corresponds to a rating scale.

Recruitment:

Subjects were recruited from the sample of participants in the PMS Symptom Monitoring protocol (COUHES 2994; CRC 522). Only those subjects that reported mild to severe PMS symptoms in their luteal phase and no symptoms during their follicular phase were asked to participate in this protocol.

Protections Against Risks:

The data obtained by the interactive voices response was stored in a Microsoft SQL-Server 2000 database. No personally identifiable information is stored in the database. The system is accessible only by using a pre-assigned subject number and personal identification number (PIN). The Principal Investigator was provided with a list of pre-assigned subject ID's and PIN's for use in the study. The principal investigator maintained a confidential log that identifies the assignment of subject numbers and PIN's, and this information was made available to the CRC staff and the nurse practitioner in case it became necessary to identify the subject.

Additional Protections Against Risks (Both Studies):

The Principal Investigator assures the confidentiality of all the research data. The research results will be kept in a secure location and access limited to the Principal Investigator and research staff. Each subject will have a medical record at the CRC. This record will be protected in compliance with the MIT Medical confidentiality policies.

### EXAMPLE 1

Number of Observations Per Subject/Intervention Combination.

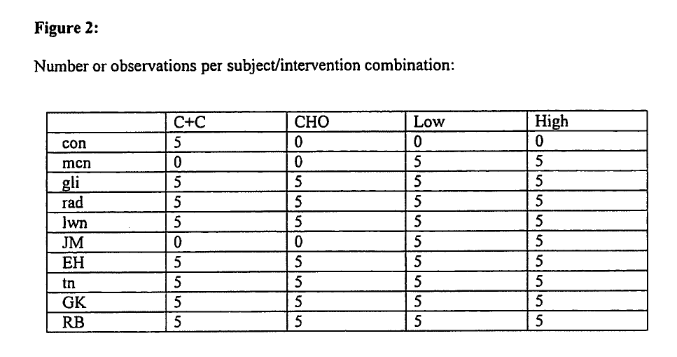

(FIG. 2). Subjects con, mcn, and JM had incomplete data sets, so they were removed from the rest of this analysis.

### EXAMPLE 2

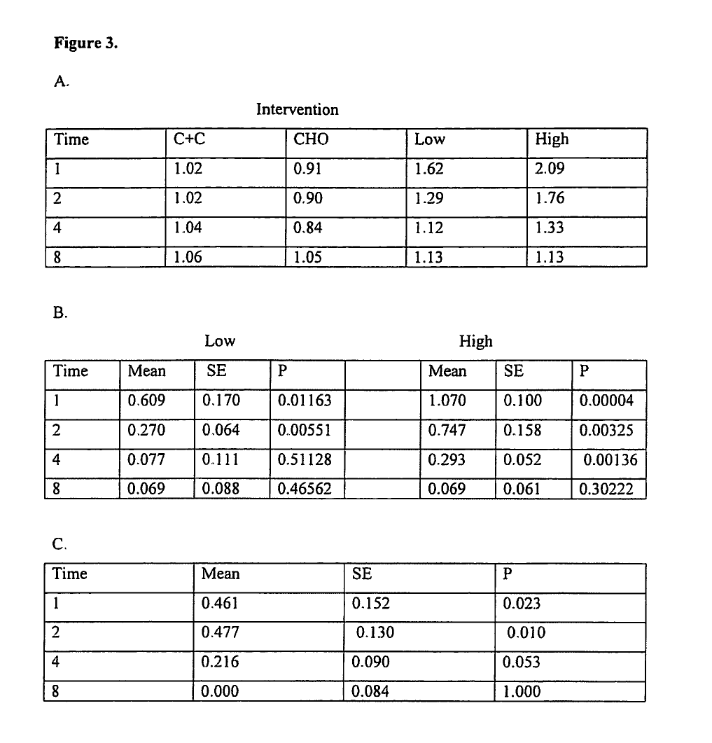

Mean treatment/baseline ratios were determined for plasma tryptophan levels (7 subjects). The results are depicted in (FIG. 3A). For each level of alpha-lac (Low, High) and each time point (1, 2, 4, 8 hrs), the mean difference between tryptophan/baseline ratios for alpha-lac and C+C are given below, along with standard errors of the mean differences and p-values from paired two-tailed t-tests (B). Adjusting for the 4 comparisons, a statistically significant difference can be claimed if P<0.05/4=0.0125.

It appears that there is a significant increase in tryptophan for alpha-lac compared with placebo early (1 h, 2 h), and that tryptophan has dropped to a level comparable with C+C by 4 hrs (“Low” alpha-lac), or 8 hrs (“High” alpha-lac). This demonstrates a dose effect.

With paired t-tests, the difference of the (alpha-lac−C+C) differences (C): “High” alpha-lac−“Low” alpha-lac was tested. These data suggest a statistically significant difference between alpha-lac+carbohydrates and casein+carbohydrates in their ability to raise plasma tryptophan levels.

### EXAMPLE 3

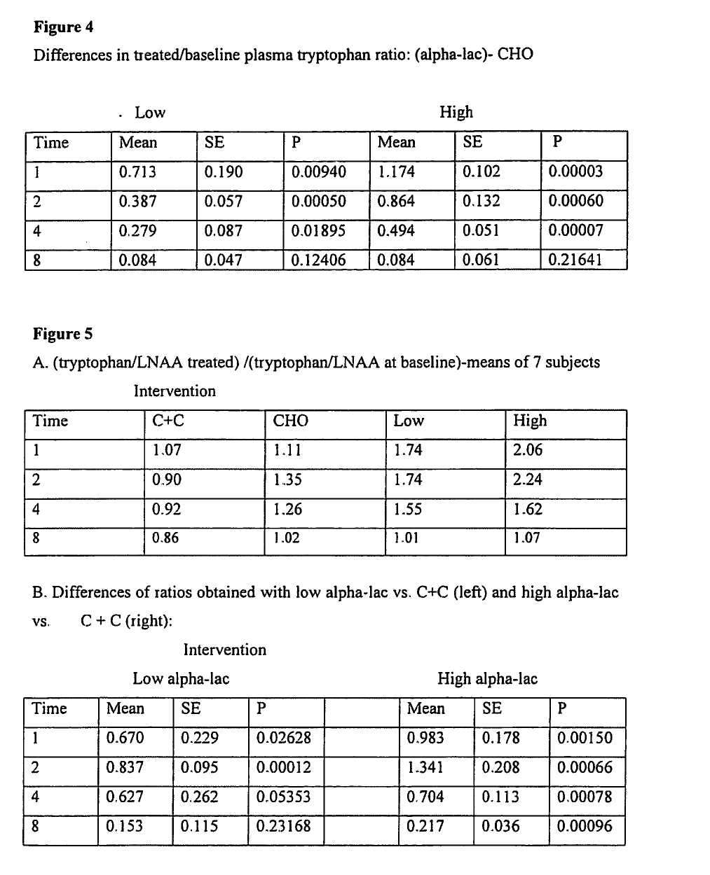

Depicted are the (alpha-lac−CHO) comparisons (FIG. 4). (The differences for the dose effect are omitted, because they are independent of the control, and so are identical to the values given above.) The pattern is as in Example 2, but the differences are greater and hence more statistically significant. By 8 hrs the tryptophan levels are comparable to CHO for both levels of alpha-lac. By 4 hrs the tryptophan level difference is almost, but not quite, statistically significant for the low alpha-lac dose, but still highly significant for the high dose.

### EXAMPLE 4

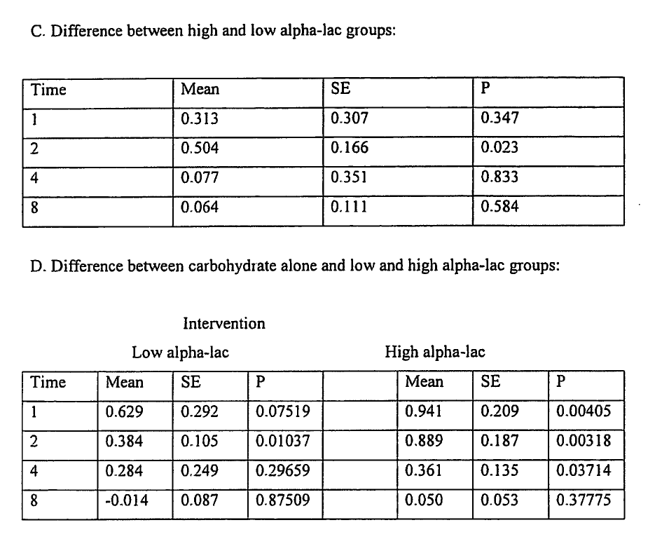

In this case, the treated/baseline ratio of the tryptophan/LNAA ratio was calculated, and the same analysis performed as for plasma tryptophan levels in Examples 2 and 3. The general pattern of the results (depicted in FIG. 5) is rather similar to the analysis (Examples 2 and 3). (A) Treated/baseline ratios of the tryptophan/LNAA ratios for the 4 groups. (B). Differences between ratios in casein+carbohydrate group and of low and high alpha-lac groups. (C) Differences between low and high alpha-lac groups. (D) Differences between ratios in carbohydrate alone group and low and high alpha-lac groups.

Perhaps the alpha-lac-high dose tryptophan/LNAA ratio does not drop to the level for C+C as quickly as did the corresponding difference for tryptophan above: after 8 hours, the difference remains highly significant. The evidence for a dose effect is also less clear for this response than was the case above, but the trend in the mean differences does demonstrate a dose effect. Again, it might be possible to show an effect with a larger number of subjects.

1. A composition comprising an amount of a carbohydrate, which has a high glycemic index, and an amount of alpha-lactalbumin protein.

2. The composition of claim 1, wherein said amount of a carbohydrate is up to 50 grams, and said amount of an alpha-lactalbumin protein is about 1-20 grams.

3. The composition of claim 1 in the form of an extruded bar.

4. The composition of claim 1 in the form of a powder.

5. The composition of claim 1 in the form of a food or beverage.

6. The composition of claim 1 in the form of a pharmaceutical composition.

7. A method of treating pain, comprising the steps of administering a composition comprising an amount of a carbohydrate which has a high glycemic index, and an amount of alpha-lactalbumin protein, thereby treating said pain.

8. The method of treating pain of claim 7, wherein said composition comprises about up to 50 grams of said carbohydrate which has a high glycemic index; and about 1-20 grams of said alpha-lactalbumin protein.

9. The method of treating pain of claim 7, wherein said pain is associated with a pre-menstrual syndrome.

10. The method of treating pain of claim 7, wherein said pain is associated with a menopause.

11. A method of ameliorating pain, comprising the steps of administering a composition comprising an amount of a carbohydrate which has a high glycemic index, and an amount of alpha-lactalbumin protein, thereby ameliorating said pain.

12. The method of ameliorating pain of claim 11, wherein said composition comprises about up to 50 grams of said carbohydrate which has a high glycemic index; and about 1-20 grams of said alpha-lactalbumin protein.

13. The method of ameliorating pain of claim 11, wherein said pain is associated with a pre-menstrual syndrome.

14. The method of ameliorating pain of claim 11, wherein said pain is associated with a menopause.

15. A method of treating increased appetite associated with a pre-menstrual syndrome, comprising the steps of administrating an amount of a carbohydrate which has a high glycemic index, and an amount of alpha-lactalbumin protein, thereby treating increased appetite associated with pre-menstrual syndrome.

16. The method of treating increased appetite associated with a pre-menstrual syndrome of claim 15, wherein said composition comprises about up to 50 grams of said carbohydrate which has a high glycemic index; and about 1-20 grams of said alpha-lactalbumin protein.

17. A method of ameliorating increased appetite associated with a pre-menstrual syndrome, comprising the steps of administrating an amount of a carbohydrate which has a high glycemic index, and an amount of alpha-lactalbumin protein, thereby ameliorating increased appetite associated with pre-menstrual syndrome.

18. The method of ameliorating increased appetite associated with a pre-menstrual syndrome of claim 17, wherein said composition comprises about up to 50 grams of said carbohydrate which has a high glycemic index; and about 1-20 grams of said alpha-lactalbumin protein.

19. A method of treating mood symptoms associated with a pre-menstrual syndrome, comprising the steps of administrating an amount of a carbohydrate which has a high glycemic index, and an amount of alpha-lactalbumin protein, thereby treating mood symptoms associated with pre-menstrual syndrome.

20. The method of treating mood symptoms associated with a pre-menstrual syndrome of claim 19, wherein said composition comprises about up to 50 grams of said carbohydrate which has a high glycemic index; and about 1-20 grams of said alpha-lactalbumin protein.

21. A method of ameliorating mood symptoms associated with a pre-menstrual syndrome, comprising the steps of administrating an amount of a carbohydrate which has a high glycemic index, and an amount of alpha-lactalbumin protein, thereby ameliorating mood symptoms associated with pre-menstrual syndrome.

22. The method of ameliorating mood symptoms associated with a pre-menstrual syndrome of claim 21, wherein said composition comprises about up to 50 grams of said carbohydrate which has a high glycemic index; and about 1-20 grams of said alpha-lactalbumin protein.

23. A method of ameliorating a pre-menstrual syndrome, comprising the steps of administrating an amount of a carbohydrate which has a high glycemic index, and an amount of alpha-lactalbumin protein, thereby ameliorating pre-menstrual syndrome.

24. The method of ameliorating a pre-menstrual syndrome of claim 23, wherein said composition comprises about up to 50 grams of said carbohydrate which has a high glycemic index; and about 1-20 grams of said alpha-lactalbumin protein.

25. A method of treating an insomnia associated with a pre-menstrual syndrome, comprising the steps of administrating an amount of a carbohydrate which has a high glycemic index, and an amount of alpha-lactalbumin protein, thereby treating insomnia associated with pre-menstrual syndrome.

26. The method of treating an insomnia associated with a pre-menstrual syndrome of claim 25, wherein said composition comprises about up to 50 grams of said carbohydrate which has a high glycemic index; and about 1-20 grams of said alpha-lactalbumin protein.

27. A method of ameliorating an insomnia associated with a pre-menstrual syndrome, comprising the steps of administrating an amount of a carbohydrate which has a high glycemic index, and an amount of alpha-lactalbumin protein, thereby ameliorating insomnia associated with pie-menstrual syndrome.

28. The method of ameliorating an insomnia associated with a pre-menstrual syndrome of claim 27, wherein said composition comprises about up to 50 grams of said carbohydrate which has a high glycemic index; and about 1-20 grams of said alpha-lactalbumin protein.
